(19) United States
(12) Patent Application Publication
(10) Pub. No.: US 2008/0267937 A1
(43) Pub. Date: Oct. 30, 2008

(54) Compositions for Preventing Plant Disease Comprising Bacillus Subtilis Kccm 10639 or Kccm 10640 and Methods of Preventing Plant Disease by Using Them

(75) Inventors: Kyu Jin Yum, Gyeonggi-Do (KR); Young Jin Koh, Jeollanam-Do (KR); Jae Seoun Hur, Jeollanam-Do (KR); Jae Wook Cha, Gyeonggi-Do (KR)

Correspondence Address: CLARK & ELBING LLP, 101 FEDERAL STREET, BOSTON, MA, 02110, US

(73) Assignee: Kyu Jin Yum, Gwacheon Si (KR)

(21) Appl. No.: 12/089,490

(22) PCT Filed: Oct. 2, 2006
(86) PCT No.: PCT/KR2006/003990

§ 371 Date: Apr. 30, 2008

(30) Foreign Application Priority Data
Oct. 7, 2005 (KR) 10-2005-0094199

(51) Int. Cl. A01N 63/00; C12N 1/20; A01P 15/00

(52) U.S. Cl. 424/93.462; 435/252.5

(57) ABSTRACT

The present invention relates to a composition for controlling plant diseases, which comprises novel bacterial strain Bacillus strain KCCM 10639 or KCCM 10640, and a method for controlling plant diseases using said bacterial strain. More particularly, the invention relates to a pure culture of novel bacterial strain Bacillus subtilis KCCM 10639, KCCM 10640 or a mixture thereof, a microbial formulation containing said pure culture as an active ingredient, and a method for controlling plant diseases using said pure strain or bacterial microorganism.

### TECHNICAL FIELD

Recently, many researchers have made an effort to change soil microflora to provide advantages for plant growth and human health. Bacterization means inoculating a microbial culture into a plant growth site, and such inoculation sometimes results in a considerable increase in plant yield, but it is not yet clear what the inoculated microorganisms play in the rhizosphere of plants. Microorganisms include microorganisms that stably survive and grow in the rhizosphere during the plant growth period to maintain the density thereof, and microorganisms that are excluded by indigenous rhizosphere microorganisms so that they fail to colonize the rhizosphere. Herein, the microorganisms that stably colonize and grow around the root are called rhizosphere microorganisms.

The root colonization of microorganisms is a very positive process, in which microorganisms survive on the seed surface or in soil, while they grow using seed exudates rich in carbohydrates and amino acids, are attached to the root surface and continue to move and grow along the root. Also, the rhizosphere microorganisms may be passively moved up to the bottom of the root by watering.

Typical examples of the rhizosphere microorganisms include Pseudomonas, Azotobacter, Bacillus and the like, and the rhizosphere microorganisms have rapid growth rate, are motile, and tend to prefer root exudates. Microorganisms that have advantageous effects on plants are called “plant growth-promoting rhizosphere microorganisms”. In old times, the advantageous effects of the plant growth-promoting rhizoshpere microorganisms were recognized only on root crops, including radish, potato and sugar cane, but the positive effects thereof have recently been recognized on various crops, including oat, bean, cotton, peanut, balsam, rice and vegetables.

The plant growth-promoting effect and disease control effect of microbial inoculation should be recognized as both sides of a coin. In other words, it has been found by many researchers that microorganisms having the growth-promoting effect also have the disease-inhibitory effect, and microorganisms having the disease-inhibitory effect also promote plant growth. For example, there are research results that indicate that some microorganisms (Pseudomonas bacteria) recognized to have the plant growth-promoting effect reduce the density of harmful bacteria and fungi in the rhizosphere to reduce plant diseases, thus promoting plant growth.

In other words, most plant growth-promoting rhizosphere microorganisms, the action mechanism of which is known, are known to indirectly promote crop growth by controlling harmful rhizosphere microorganisms.

Meanwhile, there is also a case where rhizosphere microorganisms directly promote plant growth by producing plant growth-promoting substances (physiologically active substances). In other words, these physiologically active substances are known to exhibit the effects of promoting root hairs, roots and stems by promoting the absorption of nutrients into the roots. As a result, the plant growth-promoting rhizosphere microorganisms show the growth-promoting effect by plant disease control, and also produce physiologically active special substances to change the physiology of plants so as to increase the self-defense ability of the plants against the attack of pathogens, thus showing the disease control effect.

The biological control mechanisms of rhizosphere microorganisms are known to be attributable to antibacterial activity, competition, lysis, specific nutrient exhaustion, cyanide production and the like.

In the case of antibacterial activity, rhizosphere microorganisms recognized to have antibacterial activity (antagonistic activity) against pathogenic bacteria in laboratories do not necessarily exhibit the same antibacterial activity in crop growing fields, but in most cases, show a deep correlation between the results in laboratories and the results in crop growing fields.

Although the antibacterial activity of the rhizosphere microorganisms is known to be caused by the antibiotics and siderophores secreted from the rhizosphere microorganisms, the major effects of the microorganisms on the control of disease-causing organisms are caused mainly by antibiotics. For example, some plant growth-promoting rhizosphere microorganisms, when administered to wheat growing soil, secrete a potent antibiotic, called “phenazine”, around the wheat root, to inhibit pathogenic bacteria, thus showing the effects of preventing plant diseases and increasing production yield.

As used herein, the term “competition” refers to obtaining the plant disease-inhibitory effect by making pathogenic bacteria impotent through the competition between rhizosphere microorganisms and pathogenic bacteria for rhizosphere nutrients and disease-sensitive root sites. The plant growth-promoting rhizosphere microorganisms (Pseudomonas bacteria) ingest various nutrients at a rapid rate to exhaust nutrients to be used by pathogenic bacteria. Specifically, pathogenic bacteria will invade a specific root site to cause diseases; however, the plant growth-promoting rhizosphere microorganisms preferentially colonize the specific site to prevent pathogenic bacteria from invading the root, thus preventing the occurrence of diseases.

As used herein, the term “lysis” refers to killing pathogenic fungi by the action of fungus-destroying enzymes produced by the plant growth-promoting rhizosphere microorganisms. In other words, the plant growth-promoting rhizosphere microorganisms produce a fungal cell wall-lysing enzyme, called “chitinase”, to destroy the cells of pathogenic fungi (Pythium, etc.), thus showing the effects of preventing plant diseases and increasing production yield.

Although iron, a microelement, is an element essential for the growth of microorganisms, it cannot be used directly in microorganisms, because it is present mainly as insoluble trivalent iron. To absorb and utilize the insoluble iron, microorganisms produce a substance, called “siderophore”, to make chelate compounds having siderophore-iron bound thereto. The plant growth-promoting rhizosphere microorganisms produce a large amount or good function of siderophore to use iron at rapid rate, so that they exhaust harmful microorganisms (including pathogenic bacteria) in the rhizosphere, and can thus prevent pathogenic bacteria from growing and invading the roots.

Many rhizoshpere microorganisms make cyanides, which help to control pathogenic bacteria. Specifically, the cyanides can cause fatal damage to pathogenic bacteria in the rhizosphere to suppress the growth of the pathogenic bacteria.

### BACKGROUND ART

Korean Patent Application Nos. 1998-0012807, 2001-0063465 and 2003-0079546 disclose methods for controlling plant diseases using Bacillus subtilis. A bacillus strain having an insecticidal effect against Nematoda is disclosed in Korean Patent Application Nos. 2002-004324 and 2002-004325, and a bacillus strain having an insecticidal effect against insects is disclosed in Korean Patent Application Nos. 2002-0017167 and 2004-7007871. Also, Korean Patent Application No. 2003-0005335 discloses a microbial agricultural chemical comprising Bacillus lentimobs, and a mutant strain of said microorganism.

### TECHNICAL PROBLEM

It is an object of the present invention to provide novel bacterial strain Bacillus subtilis KCCM-10639 or KCCM 10640 for controlling plant diseases.

Another object of the present invention is to provide novel bacterial strain Bacillus subtilis KCCM-10639 or KCCM 10640 effective for the prevention of turfgrass diseases.

Still another object of the present invention is to provide a microbial formulation for controlling plant diseases, which contains, as an active ingredient, a pure culture of novel bacterial strain Bacillus subtilis KCCM-10639 and/or KCCM 10640, as well as a method for controlling plant diseases using the microbial formulation.

### TECHNICAL SOLUTION

The present invention provides novel bacterial strain Bacillus subtilis KCCM-10639 and KCCM 10640 for promoting the growth of plants.

Also, the present invention provides a microbial formulation for controlling plant diseases, which contains, as an active ingredient, a pure culture of novel bacterial strain Bacillus subtilis KCCM-10639 and/or KCCM 10640.

### ADVANTAGEOUS EFFECTS

The novel bacterial strain novel strain Bacillus subtilis KCCM-10639 and KCCM 10640 according to the present invention has an excellent effect of inhibiting the occurrence of plant diseases. Particularly, the inventive microorganisms form resistant spores, so that a microbial formulation is easily prepared from the microorganisms and biologically stable. Also, the novel bacterial strain novel strain Bacillus subtilis KCCM-10639 and KCCM 10640 show resistance to agricultural chemicals, which are currently frequently used, and thus can be used alternately or simultaneously with the agricultural chemicals. Furthermore, the inventive bacterial strains have an excellent ability to grow in various disease-causing conditions, and show an excellent ability to colonize soil. Accordingly, the microbial formulation according to the present invention has an excellent ability to control plant diseases, particularly turfgrass diseases.

### BRIEF DESCRIPTION OF THE DRAWINGS

FIG. 1 shows an electron microscope photograph at 3,500×(A) and optical microscope photograph (B) at 400× of Bacillus subtilis KCCM 10639. Resistant spore portions are indicated as circles.

FIG. 2 is an electron microscope photograph of Bacillus subtilis KCCM 10640. (A): 5,000×; and (B): 10,000×.

FIG. 3 shows the antagonistic effect of Bacillus subtilis KCCM 10639 against Rhizoctonia solani. The photographs of FIG. 3 were taken after inoculating KCCM 10639 into the center of a plate in a linear form, placing on both ends of the plate an agar plate having Rhizoctonia solani grown thereon, and incubating the plates for 2 days.

FIG. 4 shows the antagonistic effect of Bacillus subtilis KCCM 10639 against Pythium sp. The left side shows a control group, and the right side shows that the growth of Pythium sp. is inhibited when KCCM 10639 is inoculated into the center of the plate in a linear form.

FIG. 5 shows the antibacterial activity of Bacillus subtilis KCCM 10640 against Rhizoctonia solani. (A): inoculated with Bacillus subtilis KCCM10640; and (B): a control group.

FIG. 6 shows the antibacterial activity of Bacillus subtilis KCCM 10640 against Pythium sp. (A): inoculated with Bacillus subtilis KCCM10640; and (B): a control group.

FIG. 7 shows an antagonistic effect against Rhizoctonia cerealis. (A): Bacillus subtilis KCCM 10639; and (B): Bacillus subtilis KCCM 10640.

FIG. 8 shows that a microbial formulation has no plant pathogenicity.

FIG. 9 shows the effect of the microbial formulation on the inhibition of turfgrass diseases.

FIG. 10 shows that the microbial formulation has the effect of inhibiting the development of diseases when it is used to treat soil contaminated with pathogenic bacteria.

### BEST MODE FOR CARRYING OUT THE INVENTION

The present invention provides novel bacterial strain Bacillus subtilis KCCM-10639 and KCCM 10640.

Also, the present invention provides a microbial formulation for controlling plant diseases, which contains, as an active ingredient, a pure culture of novel bacterial strain Bacillus subtilis KCCM-10639 and/or KCCM 10640.

The Bacillus subtilis strain according to the present invention can be used as a microbial for the control of plant diseases by mixing the strain itself, or a culture, extract or spore thereof with a carrier to formulate it into powers, pellets, granules or solutions. Herein, the carrier can be selected from the group consisting of water, white carbon, kaolin, zeolite and the like.

The microbial formulation can be used to treat either soil having plants growing thereon or the surface of the growing plant, thus preventing the inhibition of plant growth, caused by plant diseases, and the resulting plant apoptosis.

The present invention provides a method for controlling plant diseases using said microbial formulation.

Hereinafter, the present invention will be described in further detail with reference to examples. It is to be understood, however, that these examples are for illustrative purposes only and are not to be construed to limit the scope of the present invention.

### MODE FOR CARRYING OUT THE INVENTION

### Example 1

### Isolation, Identification and Characterization of Bacterial Strain

Novel bacterial strain Bacillus subtilis KCCM-10639 or KCCM 10640 was selected from the topsoil layer (about 10 cm) of the green and fairway of golf clubs present nationwide in Korea. For this purpose, bacterial strains forming resistant spores and showing excellent antibacterial activity were selected. Also, bacterial strains showing excellent resistance to existing agricultural chemicals were selected.

Among the selected microbial strains, microorganisms having excellent effects were isolated and identified, and as a result, identified as two kinds of Bacillus subtilis strains. The two kinds of Bacillus subtilis strains were deposited in Korean Culture Center of Microorganisms (KCCM) on Dec. 28, 2004, and were assigned accession numbers KCCM-10639 and KCCM 10640, respectively.

Common Characteristics of Novel Bacterial Strains KCCM 10639 and KCCM 10640

(1) These strains are indigenous microbial strains, which live in the rhizosphere and phyllosphere of plants in golf clubs.

(2) They are resistant spore-forming strains, so that a microbial formulation is easily prepared from the strains and is biologically stable.

(3) They are not killed by existing agricultural chemicals so that they can be used alternately with the agricultural chemicals.

(4) They have an excellent antibacterial activity of biologically controlling plant diseases.

(5) They have an excellent activity to grow in various disease-causing conditions.

(6) They have the excellent ability to survive and grow using cut turfgrass as feed.

(7) They have an excellent ability to colonize soil.

1. Cultural Characteristics of Novel Strains KCCM-10639 and KCCM-10640

These bacterial strains are cultured in PDA (300 g potato extract, 20 g glucose, 15 g agar, and 1 L distilled water), and a pH suitable for the growth thereof is pH 5-9, and the optimal pH for the growth thereof is about 7. Also, a temperature suitable for the growth thereof is 15-30° C., and the optimal temperature for the growth thereof is about 25° C. They grow in highly aerobic conditions, and the production of the resistant spores thereof is induced within 48 hours of culture.

2. Morphological Characteristics

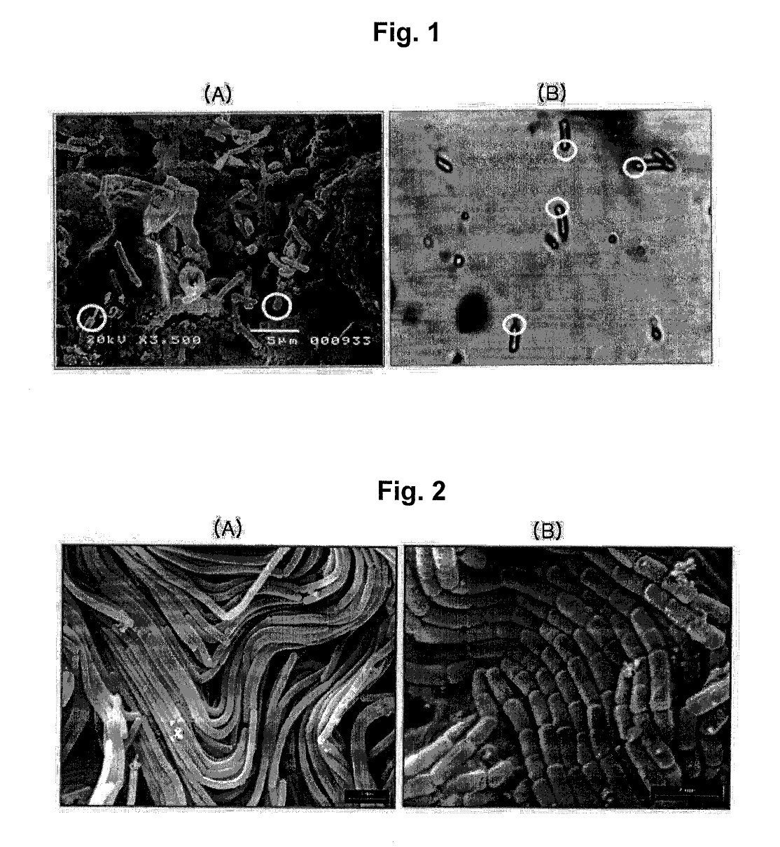

They have a rod shape, produce resistant spores, have the ability to hydrolyze starch, and are motile. FIG. 1 shows an electron microscope photograph (A) and optical microscope photograph (B) of the novel bacterial strain Bacillus subtilis KCCM 10639. In FIG. 1, portions corresponding to resistant spores are indicated as circles. FIG. 2 is an electron microscope photograph of the novel bacterial strain Bacillus subtilis KCCM 10640 (A: 5,000×; and B: 10,000×).

3. Classification

The 16s rDNA base sequence analysis of the bacterial strains was carried out, and as a result, these strains showed a homology of 99.9 to a Bacillus subtilis rDNA sequence in NCBI, and thus were identified as Bacillus subtilis.

### Example 2

### Examination of Effects of Controlling Plant Diseases

The inventive bacterial strains were tested for the effects of inhibiting Rhizoctonia diseases (large patch disease, brown patch disease, and spring dead spot) and Pythium diseases (Pythium blight), which are turfgrass diseases occurring in the green and fairway of golf clubs.

Rhizoctonia solani was used to test the effect of controlling Rhizoctonia diseases, and Pythium sp. was used to test the effect of controlling Pythium diseases. A flame-sterilized loop was lightly stained with the cultured antagonistic bacteria, and the content of nutrient broth agar was slightly rubbed 2-3 times with the stained loop. The target pathogenic bacteria Rhizoctonia spp. and Pythium sp. were detached as plates having a diameter of 8 mm, and were laid on both sides of the NB medium. After the bacteria were cultured at 25° C. for 2-3 days, and the antagonistic effect of the inventive strains against each of the target pathogenic strains was observed.

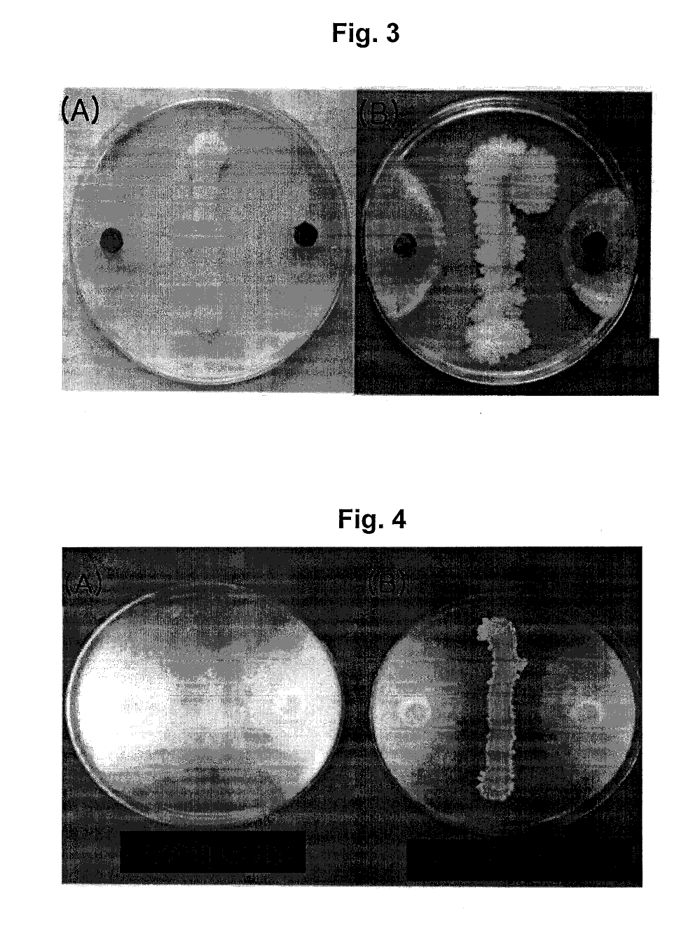

FIG. 3 shows the antagonistic effect of Bacillus subtilis KCCM 10639 against Rhizoctonia solani. The photographs of FIG. 3 were taken after inoculating KCCM 10639 into the center of a plate in a linear form, placing on both ends of the plate an agar plate having Rhizoctonia solani grown thereon, and incubating the bacteria for 2 days.

FIG. 4 shows the antagonistic effect of Bacillus subtilis KCCM 10639 against Pythium sp. In FIG. 4, the left side represents a control group, and the right side shows that the growth of Pythium sp. was inhibited when KCCM 10639 was inoculated into the center of the plate in a linear form.

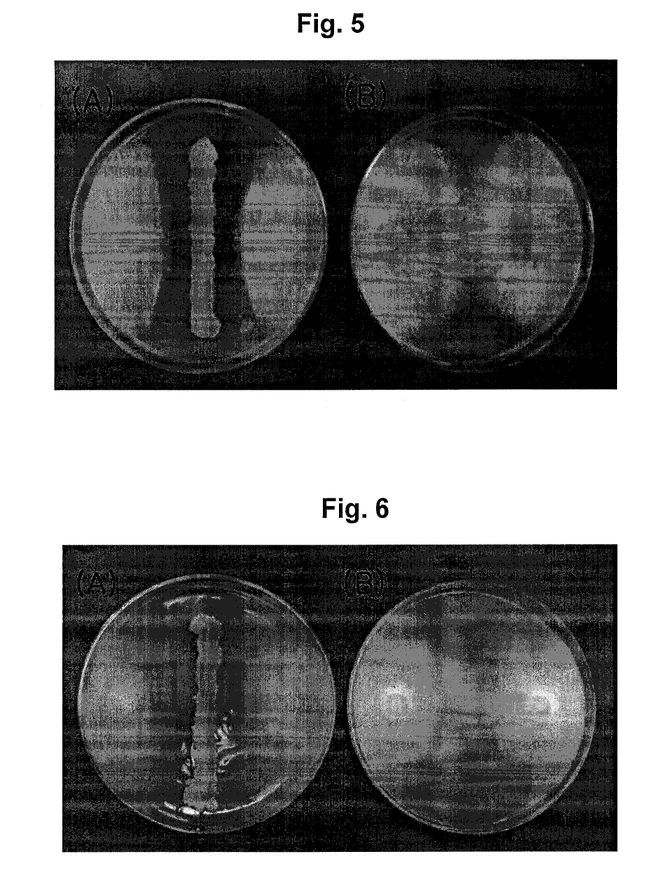

FIG. 5 shows the antibacterial activity of Bacillus subtilis KCCM 10640 against Rhizoctonia solani. In FIG. 5, (A) shows a case inoculated with Bacillus subtilis KCCM 10640, and (B) is a control group.

FIG. 6 shows the antibacterial activity of Bacillus subtilis KCCM 10640 against Pythium sp. In FIG. 6, (A) shows a case inoculated with Bacillus subtilis KCCM10640, and (B) is a control group.

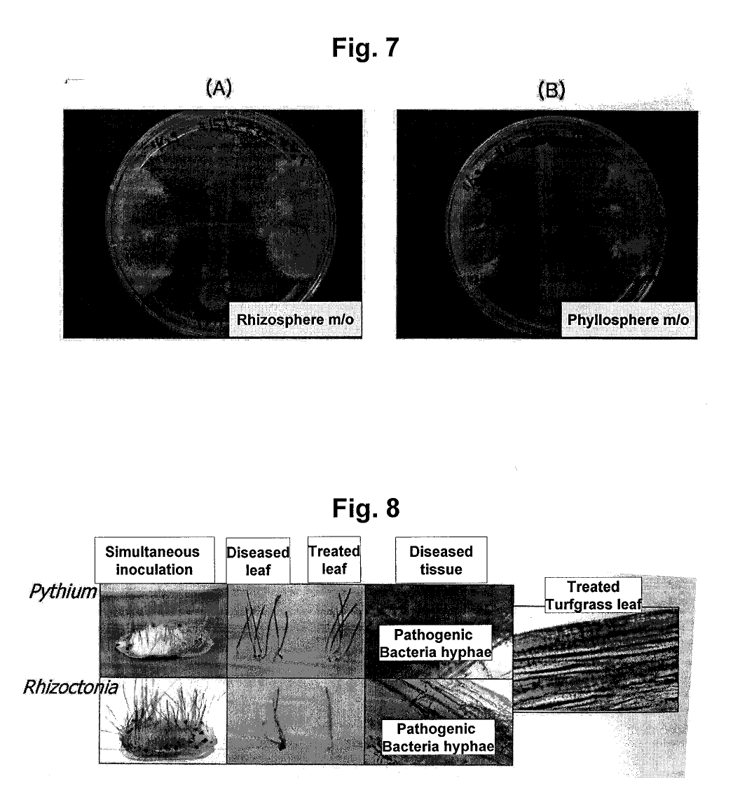

FIG. 7 shows antagonistic effects against Rhizoctonia cerealis. In FIG. 7, (A): Bacillus subtilis KCCM 10639, and (B): Bacillus subtilis KCCM 10640.

### Example 3

### Examination of Resistance to Prior Fungicides (Agricultural Chemicals)

(1) Experimental Method

(i) N.A (nutrient broth agar) medium was prepared 2-3 days before the experiment.

(ii) Sterilized water (distilled water) was prepared. Then, agricultural chemicals were dissolved in sterilized water (distilled water) in recommended amounts.

(iii) 100 μl of the solution of the step (ii) was absorbed into filter paper, followed by drying. Then, the filter paper was placed on 100 μl of smear medium. Then, the medium was sealed and incubated at 25° C. for 2 days. As a control group, distilled water was used.

(iv) The test results were observed.

(v) Whether a clear zone (growth-inhibitory zone) was produced around the paper disc was determined on the basis of whether the bacteria grew.

(vi) When the clear zone was not produced, the antagonistic strain was determined to have resistance to the agricultural chemicals, and when the clear zone was produced, the antagonistic strain was determined to be influenced by the agricultural chemicals. In this case, the diameter of the clear zone was measured and recorded.

(2) Results

The novel strains KCCM 10639 and KCCM 10640 showed resistance to the prior fungicides. The prior fungicides and the resistance of the novel strains to the fungicides are shown in Table 1.

TABLE 1

| | | | | Concentration |
|---|---|---|---|---|
| Active ingredient | Products | Trademark | Resistance | used (/100 ml) |
| Antibiotic | Polyoxin D, | Youngil-bio | Strong | 0.2 g |
| | wettable powder | | | |
| Pyrimidine | Fenarimol, | Fenarimol | Strong | 33.5 μl |
| | emulsifiable | | | |
| | concentrate | | | |
| Triazole | Tebuconazole, | Silbaco | Strong | 0.05 g |
| | wettable powder | | | |
| Triazole | Tebuconazole, | Horicoor | Middle- | 50 μl |
| | emulsifiable | | strong | |
| | concentrate | | | |
| Carbamate | Thiophanate, | Topsinmzopnate- | Strong | 0.065 g |
| | wettable powder | M | | |
| Isoxazole | Azzigaren, | Aoneipcodan | Strong | 200 μl |
| | wettable powder | | | |
| Organophosphorus | Tros, | Rizolex | Strong | 0.2 g |
| | wettable powder | | | |
| Organophosphorus | fosetyl-Al, | Aliette | Strong | 0.2 g |
| | wettable powder | | | |
| Organosulfur | Propineb, | Anthracol | Middle- | 0.2 g |
| | wettable powder | | strong | |
| Organochlorine | Safrole, | — | Middle- | 100 μl |
| | emulsifiable | | strong | |
| | concentrate | | | |
| Urine | Pencycuron, | Moongobaksa | Strong | 0.1 g |
| | wettable powder | | | |
| Acylalanine | Metalaxyl, | Lidomil | Middle- | 0.005 g |
| | wettable powder | | strong | |
| Anilide | Protonyl, | Moncut | Strong | 100 μl |
| | emulsifiable | | | |
| | concentrate | | | |
| Strobilurin | Azoxystrobin, | Heritage | Strong | 0.1 g |
| | wettable powder | | | |
| Dicarboximide | Iprodione, | Robral | Strong | 0.1 g |
| | wettable powder | | | |
| Carbamate + | Thiram, | Homai, | Weak | 0.1 g |
| dithiocarbamate | wettable powder | Kumnarac | | |
| Acylalanine + | oxadixyl | Sandophan-A | Middle- | 0.2 g |
| organosulfur | propineb, | | strong | |
| | wettable powder | | | |
| Acylalanine + | metalaxy-M, | Lidomil-MG | Weak | 0.165 g |
| dithiocarbamate | wettable powder | | | |

### Example 3

### Verification of Plant Pathogenicity

Pathogenic bacteria and the microorganisms according to the present invention were simultaneously inoculated onto turfgrass, and whether the inventive microorganisms caused diseases in the turfgrass was examined. The test results are shown in FIG. 8. As a result, it was observed that the inventive microorganisms inhibited the growth of pathogenic bacteria, but did not infect the turfgrass, suggesting that the microorganisms according to the present invention were non-pathogenic for the turfgrass and had excellent antibacterial activity.

### Example 4

### Preparation of Microbial Formulation

(1) Preparation of Liquid

(i) 8 g of nutrient broth powder was uniformly mixed with 0.5-1.0 ml of silicone oil in 1 L of water.

(ii) The mixture was autoclaved at 121° C. for 15 minutes and then inoculated with 0.1-2 ml of the antagonistic bacteria.

(iii) The inoculated solution was cultured at 25° C. for 4 days, and then the culture solution was collected.

(2) Preparation of Granule

(i) 1 L of the above-collected liquid was uniformly mixed with 5 kg of zeolite.

(ii) The mixture was dried in a granulating machine at 40° C. for 1 day, and then the dried granules were collected.

### Example 5

### Examination of Effect of Inventive Microbial Formulation on Inhibition of Turfgrass Diseases

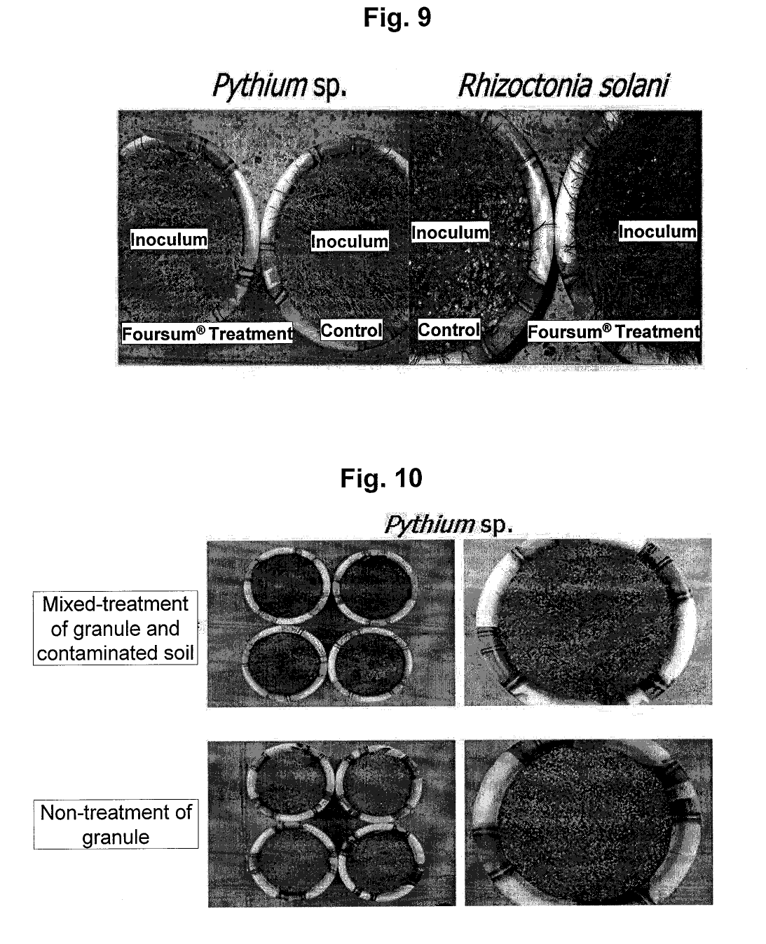

The microbial formulation prepared in Example 4 was examined for the effect of controlling plant diseases, in an artificial plant growth chamber automatic control system, the environmental factors (e.g., light intensity, temperature, humidity, etc.) of which were controlled at the same levels as in actual fields. When turfgrass was inoculated with Pythium sp. and Rhizoctonia solani and treated with the microbial formulation prepared in Example 4, the occurrence of diseases in the turfgrass was inhibited by 87-95% (see FIG. 9). Also, when soil contaminated with Pythium sp. was the microbial formulation prepared in Example 4, the occurrence of disease was inhibited by 100% (see FIG. 10).

### INDUSTRIAL APPLICABILITY

As described above, the novel bacterial strain Bacillus subtilis KCCM-10639 and KCCM 10640 according to the present invention have an excellent effect of inhibiting the occurrence of plant diseases. Particularly, the inventive microorganisms form resistant spores, so that a microbial formulation is easily prepared from the microorganisms and biologically stable. Also, the novel bacterial strain Bacillus subtilis KCCM-10639 and KCCM 10640 show resistance to agricultural chemicals, which are currently frequently used, and thus can be used alternately or simultaneously with the agricultural chemicals. Furthermore, the inventive bacterial strains have an excellent ability to grow in various disease-causing conditions, and show an excellent ability to colonize soil. Accordingly, the microbial formulation according to the present invention has an excellent ability to control plant diseases, particularly turfgrass diseases.

1. Novel bacterial strain Bacillus subtilis KCCM 10639 or KCCM 10640 for controlling plant diseases.

2. A microbial formulation for controlling plant diseases, which contains, as an active ingredient, a pure culture of the Bacillus subtilis KCCM 10639 or KCCM 10640 of claim 1 or a pure culture of a mixture thereof.

3. A method for controlling plant diseases by applying an effective amount of the Bacillus subtilis KCCM 10639 or KCCM 10640 of claim 1 or a mixture thereof to plants or soil.

4. A method for preparing a microbial formulation, comprising mixing an effective amount of the Bacillus subtilis KCCM 10639 or KCCM 10640 of claim 1 or a mixture thereof with additives.
